(19) United States
(12) Patent Application Publication
(10) Pub. No.: US 2003/0105487 A1
(43) Pub. Date: Jun. 5, 2003

(54) Hemostatic compression pad

(76) Inventors: Philip David Benz, Tigard, OR (US); Herbert J. Semler, Portland, OR (US); Gary N. Mills, Gladstone, OR (US)

Correspondence Address: PHILIP D. BENZ, 13339 SW ESSEX DR., TIGARD, OR, 97223, US

(21) Appl. No.: 10/011,828

(22) Filed: Nov. 30, 2001

(51) Int. Cl. A61B 17/00

(52) U.S. Cl. 606/201

(57) ABSTRACT

A hemostatic compression pad, for use with vascular compression devices, for application of pressure onto an area of a patient generally including a blood vessel and a wound site, such as a blood vessel puncture, during or after a medical procedure, where such procedure may be a cannulating procedure, for the purpose of controlling bleeding and achieving hemostasis. In use, the hemostatic compression pad, which is composed of at least two materials, is detachably connected to a vascular compression device and generally placed proximal to the catheter insertion site and over the blood vessel containing the cannula. The cannula is then removed from the blood vessel and pressure applied and maintained using the vascular compression device and hemostatic compression pad to compress the blood vessel for the purpose of controlling bleeding and, further, to achieve hemostasis. The improvement of this invention is its low propensity to slip laterally relative to the patient's body surface to which it is applied.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

This invention relates to a hemostatic compression pad, intended to be used with devices having the purpose of changing the rate of blood flow by means of applying compression onto an area of a patient including a wound site, such as a blood vessel puncture, and overlying a blood vessel. In particular, this invention relates to a hemostatic compression pad to be used with devices to control bleeding and achieve hemostasis in the femoral area of a groin following completion of a procedure, which involves cannulation of a femoral artery for the purpose of diagnostic catheterization or angioplasty. These devices may be collectively known as vascular compression devices. The improvement of this invention is its lower propensity to slip laterally relative to the patient's body surface to which it is applied, compared with other pressure pads.

The femoral artery is a high pressure blood vessel which requires deliberate action to achieve hemostasis (cessation of bleeding) following completion of a cannulation procedure. If a sheath is removed from, for example, the femoral artery in the groin and no attempt to achieve hemostasis is made, the patient would quickly experience rapid bleeding resulting in adverse health consequences including hypovolemia, shock, a requirement for blood transfusion, and possibly death. Application of pressure proximal and medial to a femoral artery puncture site and directly over the femoral artery is a means of achieving hemostasis, where such pressure is sufficient to slow or completely occlude blood flow in the femoral artery. This permits a clot to form which results in hemostasis at the puncture site.

One means of applying such pressure requires that an individual remain with the patient, actively pressing down with gloved hands directly over the artery and generally proximal and medial to the puncture site for a period of time which varies based on the type of procedure, the nature of the drugs administered and the patient's condition; frequently this period of time extends between 15 and 30 minutes or longer. As a result, fatigue, stiffness and pain may occur in the fingers, hands, wrist and forearms of the practitioner performing this procedure. An individual repeatedly performing this procedure over a long period of time without the aid of any assist devices could develop repetitive strain injury, for example, carpal tunnel syndrome. Also, there is the possibility that a glove could have or develop a hole, thereby allowing direct pressurized skin contact with a patient's blood.

A number of possible solutions to these problems may be collectively known as vascular compression devices, which are described in the prior art. In some of these solutions, a pressure pad is used in conjunction with said vascular compression devices to compress a portion of a patient's body surface, in turn compressing a blood vessel and subsequently resulting in hemostasis.

One possible solution is described by Semler in U.S. Pat. No. 3,779,249 and U.S. Pat. No. 5,304,186. The devices described in these patents are generally comprised of a c-clamp mechanism, to which a detachable, disk-shaped pressure pad is attached when being used on a patient. An advantage of using the c-clamp mechanism is its ability to free the hands of the attending practitioner and to provide consistent pressure during the compression period.

Another possible solution is an apparatus to facilitate sealing of arterial punctures described by Abrams, et al in U.S. Pat. No. 3,625,219, which includes a means of attaching said apparatus to the patient to which it is being applied, and an inflatable pad unit which applies and maintains compression over an artery in response to manual pumping of a pneumatic inflation device, in place of a removable pressure pad which is not inflatable.

Another possible solution is a hemostatic compressive device described by McRae, et al in U.S. Pat. No. 4,233,980 and includes an inflatable bladder and pressure plate as the means of applying and maintaining compression over a blood vessel, in place of a removable pressure pad which is not inflatable.

A notched pressure pad, which can be used with a vascular compression device such as those described by Semler, is described by Royse in U.S. Pat. No. 4,572,182. This invention includes a notch, which may be V-shaped, in the base of the disc-shaped pressure pad, for the purpose of enabling placement onto a patient's body surface prior to withdrawal of the catheter from the blood vessel. Royse contemplates the possibility of slippage of the pressure pad once it has been applied to the patient and in the specification includes, during the injection molding process by which the pressure pad is formed, a sand-blasted surface or other type of gripping surface, as a means of preventing slippage.

Another possible solution is an adjustable compress apparatus described by Freund, et al in U.S. Pat. 4,742,825 which includes screw-type adjustment mechanisms and a detachable pressure pad which is attached by a ball-and-socket connection.

Another possible solution is a radial arm quick adjusting artery clamp described by Rice in U.S. Pat. No. 5,304,201 which includes locking mechanisms and a detachable pressure pad having a means of snapping it on and off of the carrier of the clamp.

Another possible solution is an apparatus which is described by Toller in U.S. Pat. No. 5,342,388. Said apparatus is comprised of an elongated substantially cylindrically shaped handle weighing at least about one pound, an elongated rod extending outward from said handle, to which a removable, disposable, sterile pressure pad is attached when being used on a patient. A disposable sterile disk member, which can be used with said apparatus, is described by Royse in U.S. Pat. No. 4,572,182.

A haemostatic pressure pad is described by Lam in U.S. Pat. No. 5,601,596 which is capable of applying pressure simultaneously onto both the femoral artery and the femoral vein. The Lam invention is attached to a vascular compression device for its operation, and further includes a method of orienting and positioning the pressure pad during use.

The disadvantage present in all of the aforementioned prior art is that none include an effective means of controlling lateral slippage of the pressure pad relative to the patient's body surface once said pressure pad has been applied under pressure to the patient. Such slippage can result in post-catheterization complications including hematoma, pseudoaneurysm, or in extreme cases, uncontrolled bleeding from the arterial puncture site.

Pressure pads in common use, which include some of those described in the prior art, are made of generally rigid materials, for example, a plastic such as an acrylic. Flexure permitted by non-rigid materials results in deflection of the peripheral portions of the pressure pads when applied to a patient's body surface under pressure thereby diminishing the area through which pressure is applied to said body surface. This may result in inaccurate placement of the pressure pad upon the body surface, in turn resulting in failure to achieve proper compression of the blood vessel and subsequent hemostasis. The rigidity of these pressure pads is necessary to ensure that a minimum area of consistent pressure is applied when compression of a blood vessel and subsequent hemostasis is desired.

Pressure pads also may include a texture or pattern molded onto the bottom surface of the pressure pad making contact with the patient's body surface. Said textures or patterns do not prevent the pressure pads from slipping laterally relative to the patient's body surface because of the relatively low friction coefficient of the materials themselves, texturing limitations imposed by patient comfort requirements, and the injection molding process used to form the pressure pads. Where the patient is obese, body parts obtruding against members of a vascular compression device to which the pressure pad is attached as described in the prior art may cause significant lateral slippage of the pressure pad relative to the patient's body surface; even where such obtrusion is not present, patient body movement or even coughing by the patient may cause such slippage to occur.

Nothing in the other prior art, with the sole exception of Royse, acknowledges or attempts to address the slippage problem. However, in U.S. Pat. No. 4,572,182 Royse does not effectively address the slippage problem. As has been observed in clinical practice, the materials and injection molding process used in the composition of the pressure pad described by Royse result in surfaces having a relatively low coefficient of friction which permit lateral slippage of the pressure pad relative to the patient's body surface even in the presence of the sand-blasted surface to which Royse makes reference, or where other textures on the bottom surface of the pressure pad are present.

### SUMMARY OF THE INVENTION

An object of this invention is to provide an improved pressure pad for use with vascular compression devices. This invention assists a user in controlling bleeding and achieving hemostasis, through the proper, laterally stable application of pressure onto a body surface thereby compressing the lumen of an underlying blood vessel. This compression in turn reduces or halts blood flow through said blood vessel.

A further object of the invention is to assist a user in controlling bleeding and, in particular, achieving hemostasis after removing a catheter or cannula from a blood vessel by using this invention with a vascular compression device.

A further object of the invention is to provide a disposable pressure pad for use with vascular compression devices, including those described in the prior art, which provides improved stability of the pressure pad relative to a body surface of a patient on whom it is used, such improved stability comprising reduced propensity to slip laterally relative to said body surface once compression is applied.

These and other objects are achieved with this invention, a hemostatic compression pad, described herein.

In a preferred embodiment of said compression pad, the circumference of the compression pad is round, formed as a disk with an upturned perimeter and an open notch cut into its perimeter, similar to the pressure pad described by Royse in U.S. Pat. No. 4,572,182. The compression pad may alternatively take another shape which accommodates similar function; for example, the compression pad may be formed with a generally oval circumference, or as a generally flat disk-shaped or irregularly-shaped member. A connecting means is included in the compression pad, which enables connection to and removal from a vascular compression device.

An important advantage of the notch included in said preferred embodiment is that it enables proper placement of the compression pad onto said patient's body surface in proximity to a puncture site, and overlies the blood vessel for which compression is desired and enables placement onto the patient prior to pulling the sheath. In addition, it permits visualization of and access to the puncture site while compression is being applied. An important advantage of the upturned perimeter is that it enables increased comfort to the patient on whom it is applied, since it replaces hard edges which would otherwise press into the body surface of the patient.

The compression pad is composed of at least two materials. A first material, which is rigid, generally defines the compression pad's shape. The rigidity of said first material provides a minimum non-yielding area of stable compression onto the patient's body surface thereby enabling compression of the blood vessel; a compression pad made completely of a non-rigid material would yield to a patient's body surface thereby permitting only a smaller area of compression onto the body surface, providing a less reliable compression pad. A second material, which has a higher coefficient of friction than the first material, covers certain surfaces of the compression pad, thereby minimizing the opportunity for slipping relative to the surface of the patient's body, or inadvertently slipping off of the connection to a vascular compression device, or being accidentally dropped by the user depending on which surfaces are covered by said second material. In particular, the second material, for example an elastomeric polyurethane having a high coefficient of friction relative to the first material, is applied to form a friction member on at least a portion of the compression pad making contact with the patient's body surface, and may additionally include a texture or treatment on said friction member's bottom surface to further minimize the opportunity for slipping relative to the surface of the patient's body.

The compression pad is therefore comprised of a substrate composed of the first material, and of a friction member composed of the second material covering at least some of the surfaces of the compression pad, where said surfaces include at least a portion of the surface making contact with the patient's body surface. The second material is attached to the first material during the manufacture of the compression pad.

The friction member is attached to the substrate using any of several means singly or in combination with each other. Where the second material is able to adhere to the first material by means of molecular cross-linking or other bonding created during the injection-molding process in the manufacture of the compression pad, the second material can be thusly applied directly to the substrate. Alternatively the friction member may be attached to the substrate during the manufacture of the compression pad using adhesive material applied to at least one of the adjoining surfaces of each of these elements to join them together. Because the attachment of the friction member to the substrate is by means of said cross-linking or other bonding, or an adhesive, no mechanical securement is required when these attachment methods are used.

Another means of securing the friction member to the substrate employs a mechanical means of securement, whereby at least one foramen, which is in open communication with the top and bottom surfaces of the substrate, is included in the substrate. The second material, when molded onto the bottom surface of the substrate to form the friction member, also flows through the foramen and is captured on the top surface of the substrate during the injection-molding process to form a top head. The top head is composed continuously of the same second material as is present in the foramen and the friction member, therefore the friction member is mechanically secured even when pulling or lateral forces are applied to it. The top head may protrude above the level of the top surface of the substrate or may be level with it. An embodiment having a top head level with the top surface of the substrate has the foramen having a generally conical shape, with the narrow apex of the cone located at the bottom surface of the substrate and the wide portion of the cone located at the top surface of the substrate; the second material filling the foramen therefore takes on a conical shape, and being composed continuously of the same second material as the friction member mechanically secures the friction member in place. Another modification to this embodiment includes the top head generally covering a greater area on the top surface of the substrate than other embodiments previously presented and may take the form of a continuous band of the second material connecting the openings of the foramens located on the top surface of the substrate, or may cover a portion or all of the entire surface of the substrate. A modification to this approach replaces the foramen with a cavity, which is hollow and has its opening on the bottom surface of the substrate and is closed at the end opposite that where the opening is located. The second material, when molded onto the bottom surface of the substrate to form the friction member, flows into said cavity to form a post. Since the post is composed continuously of the same second material as is present in the friction member, the friction member is thereby mechanically secured. The sectional shape of the foramen and cavity may vary without substantially changing the purpose, scope and application of the invention.

Another means of securing the friction member to the substrate employs a mechanical means of securement, whereby at least one recess or cavity, which is located with its opening on the bottom surface of the substrate, is included in the substrate, but does not pass entirely through to the substrate's top surface. The second material, when molded onto the bottom surface of the substrate to form the friction member, flows into the recess forming a boss protruding upwards into the substrate. The bottom surface of the friction member extends downwards to a level below the bottom surface of the substrate. In a modification to this embodiment, the friction member may be contained entirely within the recess such that the bottom surface of the friction member is level with the bottom surface of the substrate. Since the boss is composed continuously of the same second material as is present in the friction member, the friction member is mechanically secured.

Another means of securing the friction member to the substrate employs a mechanical means of securement, whereby a groove traverses at least a portion of the circumference of the substrate on the upturned perimeter. The second material, when molded onto the bottom surface of the substrate to form the friction member which covers an area of the substrate including said groove, flows into the groove thereby forming a boss protruding into the substrate, the friction member having its top edge above the groove. Since said boss is composed continuously of the same second material as is present in the friction member, the friction member is mechanically secured by the boss located in the groove.

The hemostatic compression pad therefore achieves its objects: the compression pad provides an improved pressure pad for use with vascular compression devices to assist a user in controlling bleeding and achieving hemostasis through proper, laterally stable application of pressure onto the body surface, where such laterally stable application of pressure is achieved through the higher friction between the compression pad and the patient's body surface. This higher friction is provided by the second material forming a friction member attached to at least a portion of the surface of the compression pad; more particularly, this improvement applies when using the compression pad with vascular compression devices for the purpose of removing catheters or other cannulating devices from a patient following a vascular catheterization using the femoral approach. Since the attachment of the second material is made during the manufacturing process of the compression pad, and of a material which does not create excessive cost, the compression pad may be disposable for reasons of sterility, low cost, and user convenience.

In particular, the compression pad achieves the following benefits, relative to the prior art:

(a) Slippage of the compression pad relative to the patient's body surface is greatly minimized or eliminated;

(b) Clinical outcomes are expected to improve because puncture site complications are avoided as a result of including the friction member, composed of the second material and covering at least a portion of the surface of the compression pad, for the purpose of eliminating or greatly minimizing slippage of the compression pad relative to the patient's body surface;

(c) The compression pad may be used with at least some of the vascular compression devices described in the prior art, including the devices described by Semler.

### BRIEF DESCRIPTION OF THE DRAWINGS

FIG. 1 shows a partial side section view of an embodiment of the hemostatic compression pad applied to a body surface with a vascular compression device.

FIG. 2 shows a perspective view of an embodiment of the hemostatic compression pad.

FIG. 3A shows a perspective view of an embodiment of the hemostatic compression pad.

FIG. 3B shows a perspective view, with parts broken away, of an embodiment of the hemostatic compression pad.

FIG. 4A shows a top view of an embodiment of the hemostatic compression pad.

FIG. 4B shows a top view of an embodiment of the hemostatic compression pad.

FIG. 5A shows a bottom view of an embodiment of the hemostatic compression pad.

FIG. 5B shows a bottom view of an embodiment of the hemostatic compression pad.

FIG. 5C shows a bottom view, with parts broken away, of an embodiment of the hemostatic compression pad.

FIG. 5D shows a bottom view of an embodiment of the hemostatic compression pad.

FIG. 6A shows a section view on line 6A-6A in FIG. 4B showing a means of attaching the friction member to the substrate.

FIG. 6B shows a view similar to that shown in FIG. 6A, showing another means of attaching the friction member to the substrate.

FIG. 6C shows a view similar to that shown in FIG. 6A, showing another means of attaching the friction member to the substrate.

FIG. 6D shows a view similar to that shown in FIG. 6A, showing another means of attaching the friction member to the substrate.

FIG. 6E shows a view similar to that shown in FIG. 6A, showing another means of attaching the friction member to the substrate.

FIG. 6F shows a view similar to that shown in FIG. 6A, showing another means of attaching the friction member to the substrate.

FIG. 6G shows a section view showing another means of attaching the friction member to the substrate.

FIG. 6H shows a view similar to that shown in FIG. 6A, showing another means of attaching the friction member to the substrate.

FIG. 7A shows a perspective view of the connector of the hemostatic compression pad.

FIG. 7B shows a side section view of the connector of the hemostatic compression pad.

### DETAILED DESCRIPTION OF THE INVENTION

The hemostatic compression pad can be used with a vascular compression device on a patient to apply pressure on an area near a wound site, such as a blood vessel puncture, during or after a medical procedure which may be a cannulated procedure such as angioplasty, for the purpose of controlling the patient's bleeding and, further, of achieving hemostasis.

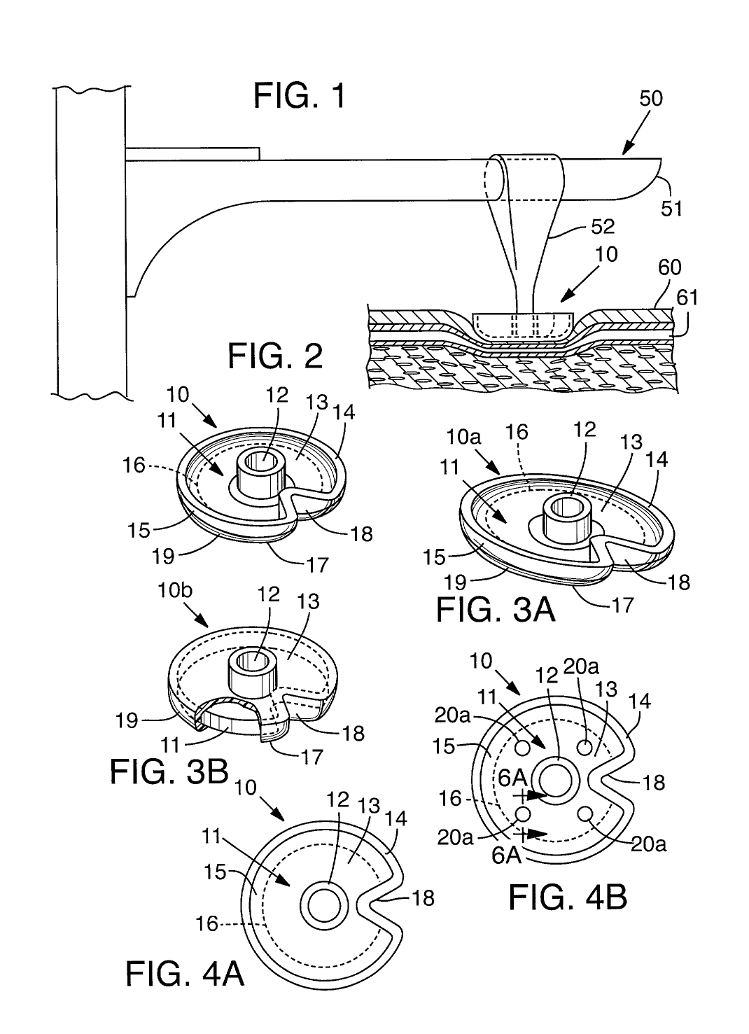

Referring to FIGS. 1-5D there is shown an invention 10, the hemostatic compression pad.

FIG. 1 shows a partial side section view generally showing application of the hemostatic compression pad 10, attached to a vascular compression device 50, to a patient, whereby said compression pad 10 is placed on the patient's body surface 60 generally over a blood vessel 61 for which control of bleeding and hemostasis is desired. When downward pressure is applied by a user to an arm 51 of the vascular compression device 50 and maintained, said downward pressure is transferred to the compression pad 10 through an arm slide 52 to which the compression pad 10 is attached, and therefrom onto the patient's body surface 60. The compression pad 10 thereby depresses the area of the body surface 60 upon which it is placed, compressing the lumen of the blood vessel 61 over which it is placed to partially or completely occlude the blood vessel 61. Such occlusion limits or completely stops the flow of blood in the blood vessel 61 during such application and maintenance of the pressure, enabling hemostasis to occur. Once hemostasis occurs the vascular compression device 50 and compression pad 10 are removed from the patient. It can be appreciated that the compression pad 10, with a vascular compression device 50 may also be used to externally compress other luminal tissue for the purpose of controlling fluid flow therein. The compression pad 10 may be used with vascular compression devices including those described in the prior art.

FIG. 2 shows a perspective view of an embodiment of the hemostatic compression pad 10. Said compression pad 10 includes a compression pad substrate 11, which is generally composed of a first material which is generally rigid, for example an acrylic. Said substrate 11 includes a connector 12, a rim 14, an upturned perimeter 15, a radius of the upturned perimeter 16, and a notch 18. The compression pad 10 further includes a flat top side 13, and a bottom side 17. In a preferred embodiment, a friction member 19 is attached to the substrate 11, whereby the friction member 19 covers at least a portion of the bottom surface of the substrate 11. The friction member 19 is composed of a second material having a high coefficient of friction compared to that of the substrate 11; an example of said second material is an elastomeric polyurethane. The shape of the circumference of the compression pad 10 is generally circular, and the notch 18 is in a V shape, with the widest portion of said V shape at the perimeter, and with the apex pointed towards the center of the compression pad 10. In this embodiment the connector 12 permits attachment to and detachment from a vascular compression device 50 by a user. Alternative shapes and sizes of the circumference and of the notch 18 of the compression pad 10 may be included without substantially changing the purpose, scope and application of the invention.

FIG. 3A shows a perspective view of an alternative embodiment of the invention, the circumference of which has a different shape than that shown in FIG. 2. The compression pad 10a is shown as having an oval-shaped circumference and includes a substrate 11, which is generally composed of a rigid material, for example an acrylic. Said substrate 11 includes a connector 12, a rim 14, an upturned perimeter 15, a radius of the upturned perimeter 16, and a notch 18. The compression pad 10a further includes a flat top side 13, and a bottom side 17. The friction member 19 is attached to the substrate 11, whereby the friction member 19 is placed on at least a portion of the bottom surface of the substrate 11. The friction member 19 is composed of a second material having a high coefficient of friction compared to that of the substrate 11; an example of said second material is an elastomeric polyurethane.

FIG. 3B shows a perspective view, with parts broken away, of an alternative embodiment of the compression pad 10b, including a substrate 11 shown in phantom, a connector 12, a top side 13, a bottom side 17, a notch 18 and friction member 19. This embodiment shows the compression pad 10b as a unitary, solid member wherein the friction member 19 covers at least substantial portions of the surfaces of the compression pad 10b. Further, the compression pad 10b is shown to have a generally circular circumference, although the shape may vary without substantially affecting the purpose, scope and application of the invention.

FIG. 4A shows a top view of the invention. The compression pad 10 includes a substrate 11, which is generally composed of a rigid material, for example an acrylic. Said substrate 11 includes a connector 12, a rim 14, an upturned perimeter 15, a radius of the upturned perimeter 16, and a notch 18. The compression pad 10 further includes a flat top side 13. Not shown in this view but included in this embodiment is the friction member 19 located on at least a portion of the bottom side 17 of the compression pad 10.

FIG. 4B shows an alternative top view of the invention in which a method of securing the friction member 19 to the substrate 11 is portrayed. The compression pad 10 includes a substrate 11, which is generally composed of a rigid material. Said substrate 11 includes a connector 12, a rim 14, an upturned perimeter 15, a radius of the upturned perimeter 16, and a notch 18. The substrate 11 also includes a foramen 21, not shown in this view, but shown in FIG. 6A in section view, the foramen 21 passing through the entire thickness of the substrate 11 and having openings on the top and bottom surfaces of the substrate 11. The compression pad 10 further includes a flat top side 13. Not shown in this view but included in this embodiment is the friction member 19 located on at least a portion of the bottom side 17 of the compression pad 10. Located on the flat top side 13 is at least one top head 20a composed continuously of the same second material used to form the friction member 19, where the second material is injection molded onto the substrate 11. The second material passes through each foramen 21 to form each top head 20a as shown in FIG. 6A in section view. Therefore, the top head 20a is formed at the same time as the friction member 19 is molded onto at least a portion of the bottom surface of the substrate 11. As shown in FIG. 6A the top head 20a includes a bottom surface at its outer perimeter which rests upon the top side of the substrate 11, and serves to mechanically secure the friction member 19 to the bottom side 17 of the compression pad 10. The top view in this FIG. 4B shows a round circumference of the top head 20a, however, the top head 20a or the foramen 21 may take different shapes without departing from the purpose, scope and application of the invention.

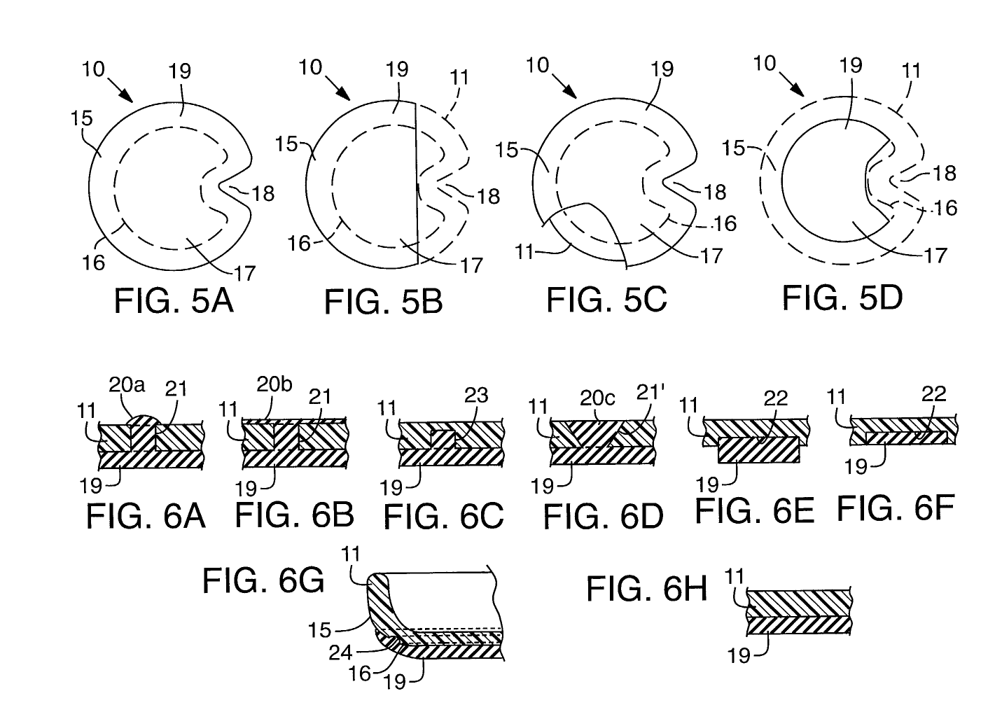

FIG. 5A shows a bottom view of an embodiment of the invention. This view shows the compression pad 10 having a generally circular circumference and including a substrate 11, a notch 18, and a friction member 19 shown in solid line, generally covering the bottom side 17, including the upturned perimeter 15. In a modified embodiment, the friction member 19 generally covers the bottom side 17, but not the upturned perimeter 15 of the compression pad 10. The compression pad includes a substrate 11, not shown in this view, composed of a generally rigid material, for example an acrylic. The friction member 19 is composed of a second material having a high coefficient of friction compared to that of the substrate 11; an example of said second material is an elastomeric material.

FIG. 5B shows a bottom view of another embodiment of the invention. This view shows the compression pad 10 having a generally circular circumference and including a substrate 11, a notch 18, and a friction member 19 shown in solid line, generally covering only a portion of the bottom side 17 of the compression pad 10 and at least a portion of the upturned perimeter 15. In a modified embodiment, the friction member 19 generally covers a portion of the bottom side 17, but not the upturned perimeter 15 of the compression pad 10.

FIG. 5C shows a bottom view, with parts broken away, of another embodiment of the invention. This view shows the compression pad 10 having a generally circular circumference and including a substrate 11, a notch 18, and a friction member 19. The friction member 19 is shown in solid line, generally covering the bottom side 17 and the top side 13, the substrate 11 located between the portions of the friction member 19 covering the bottom side 17 and top side 13.

FIG. 5D shows a bottom view of another embodiment of the invention. This view shows the compression pad 10 having a generally circular circumference and including a substrate 11, notch 18, and a friction member 19 shown in solid line, generally having a shape conforming to the shape of the circumference of the bottom side 17, and not covering the upturned perimeter 15.

Referring to FIGS. 6A-6H there are shown side section views of alternative embodiments of the invention illustrating different means of attaching the friction member 19 to the substrate 11. Each such means may be used, singly or in combination with each other, in conjunction with the embodiments of this invention described in FIGS. 1-5D. The substrate 11 may further have a texture or pattern in its bottom surface, to farther facilitate attachment of the friction member 19. FIG. 6A is a section view on line 6A-6A in FIG. 4B; FIGS. 6B-6F and FIG. 6H are views similar to that shown in FIG. 6A.

FIG. 6A shows a means of attaching the friction member 19 to the substrate 11, whereby at least one foramen 21, which is in open communication with the top surface and bottom surface of the substrate 11, is included in the substrate 11. The second material, when molded onto the bottom surface of the substrate 11 to form the friction member 19, flows through the foramen 21 and is captured on the top surface of the substrate 11 during the injection-molding process to form a top head 20a. The top head 20a includes a bottom surface at its outer perimeter which rests upon the top surface of the substrate 11. Since the top head 20a is composed continuously of the same second material as is present in the foramen 21 and the friction member 19, the friction member 19 is mechanically secured to the substrate 11 even when pulling or lateral forces are applied to it. Although the shape of the foramen 21 is shown to be generally cylindrical in this FIG. 6A, said shape may take a different form including, in profile, a cone, an I shape, a T shape, or a bell shape.

FIG. 6B shows a means of attaching the friction member 19 to the substrate 11, whereby at least one foramen 21, which is in open communication with the top surface and bottom surface of the substrate 11, is included in the substrate 11. The second material, when molded onto the bottom surface of the substrate 11 to form the friction member 19, flows through the foramen 21 and is captured on the top surface of the substrate 11 during the injection-molding process to form a top head 20b. The top head 20b includes a bottom surface which rests upon the top surface of the substrate 11. Since the top head 20b is composed continuously of the same second material as is present in the foramen 21 and the friction member 19, the friction member 19 is mechanically secured to the substrate 11. The top head 20b generally covers a greater area on the top surface of the substrate 11 than does the top head 20a shown in FIG. 6A, and may take the form of a continuous band of the second material connecting the openings of the foramens 21 located on the top surface of the substrate 11 or may cover a portion or all of the surface of the substrate 11. Although the shape of the foramen 21 is shown to be generally cylindrical in this FIG. 6B, said shape may take a different form including, in profile, a cone, a T shape, an I shape or a bell shape.

FIG. 6C shows a means of attaching the friction member 19 to the substrate 11, whereby at least one cavity 23 which is located with its opening on the bottom surface of the substrate 11, is included in the substrate 11, but does not pass entirely through to the top surface of the substrate 11. The second material, when molded onto the bottom surface of the substrate 11 to form the friction member 19, flows into the cavity 23 forming a post molded into the cavity 23. Since said post is composed continuously of the same second material as is present in the friction member 19, the friction member 19 is mechanically secured to the substrate 11. This FIG. 6C shows the shape of the post as being generally cylindrical, but the shape of the post and of the corresponding cavity 23 may be different, including an inverted cone or a T shape, without departing from the purpose, scope and application of the invention.

FIG. 6D shows a means of attaching the friction member 19 to the substrate 11, whereby at least one foramen 21′, which is in open communication with the top surface and bottom surface of the substrate 11, is included in the substrate 11. The interior of the foramen 21′ is in the general shape of a cone, with the narrowest portion of said cone located at the bottom surface of the substrate 11 and the widest portion of the cone located at the top surface of the substrate 11. The second material, when molded onto the bottom surface of the substrate 11 to form the friction member 19, flows into the foramen 21′ and being captured within the foramen 21′ during the injection-molding process forms a top head 20c, which is in the general shape of the cone defined by the shape of the foramen 21′. The top head 20c being composed continuously of the same second material as is present in the friction member 19, the friction member 19 is mechanically secured to the substrate 11. This FIG. 6D shows the shape of the top head 20c as being generally level with the top surface of the substrate 11 and in the general shape of a cone, but the shape of the top head 20c and of the corresponding foramen 21′ may be different, including radii or chamfers, without departing from the purpose, scope and application of the invention.

FIG. 6E shows a means of attaching the friction member 19 to the substrate 11, whereby at least one recess 22, which is located with its opening on the bottom surface of the substrate 11, is included in the substrate 11, but does not pass entirely through to the top surface of the substrate 11. The second material, when molded onto the bottom surface of the substrate 11 to form the friction member 19, flows into the recess 22 forming a boss protruding upwards into the substrate 11. The bottom surface of the friction member 19 extends downwards to a level below the bottom surface of the substrate 11. Since said boss is composed continuously of the same second material as is present in the friction member 19, the friction member 19 is mechanically secured to the substrate 11. Further, the sidewalls of the recess 22, although shown in this FIG. 6E to be perpendicular to the bottom surface of the substrate 11, may be at an angle to the plane formed by said bottom surface Although the portion of the friction member 19 extending to a level below the bottom surface of the substrate is shown to have straight edges in this FIG. 6E, such edges may be radiused or chamfered to provide additional comfort to the patient.

FIG. 6F shows a means of attaching the friction member 19 to the substrate 11, whereby at least one recess 22, which is located with its opening on the bottom surface of the substrate 11, is included in the substrate 11, but does not pass entirely through to the top surface of the substrate 11. The second material, when molded onto the bottom surface of the substrate 11 to form the friction member 19, flows into the recess 22 forming a boss protruding upwards into the substrate 11. The friction member 19 is contained within the recess 22 such that the bottom surface of the friction member 19 is level with the bottom surface of the substrate 11. Since said boss is composed continuously of the same second material as is present in the friction member 19, the friction member 19 is mechanically secured to the substrate 11. Further, the sidewalls of the recess 22, although shown in this FIG. 6F to be perpendicular to the bottom surface of the substrate 11, may be at an angle to the plane formed by said bottom surface.

FIG. 6G shows a means of attaching the friction member 19 to the substrate 11 in section view, whereby a groove 24, shown in dashed line, traverses at least a portion of the circumference of the substrate 11 on the upturned perimeter 15, on or above the radius of the upturned perimeter 16. The second material, when molded onto the bottom surface of the substrate 11 to form the friction member 19 which covers an area of the substrate 11 including the groove 24, flows into the groove 24 forming a boss protruding into the substrate 11. Since said boss is composed continuously of the same second material as is present in the friction member 19, the friction member 19 is mechanically held in place by the boss located in the groove 24.

FIG. 6H shows a means of attaching the friction member 19 to the substrate 11, whereby the substrate 11 is composed of a first material and the friction member 19 is composed of a second material molded onto the bottom surface of the substrate 11. Said first material and said second material have the properties of forming at least some molecular cross-linking or other bonding to create adhesion as a function of the injection molding process between the second material forming the friction member 19 and the first material forming the substrate 11. In an alternative embodiment of this method, the friction member 19 may be attached as a pre-formed element to the substrate 11 using adhesive material placed on their adjoining surfaces.

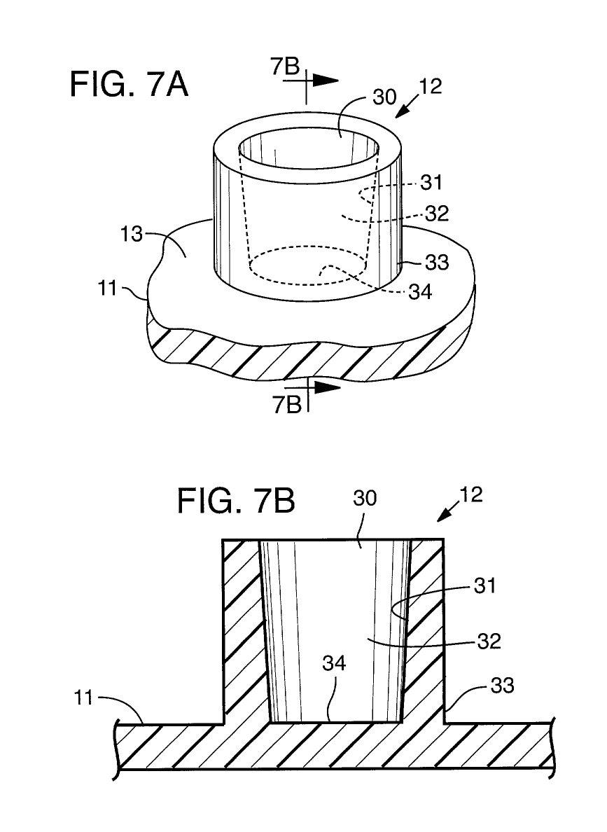

FIG. 7A shows an embodiment of the connector 12 in a perspective section view, whereby the connector 12 includes a top opening 30 having a certain diameter, an interior sidewall 31, a hollow interior 32, an exterior sidewall 33 and a closed bottom end 34. The bottom end 34 has a diameter smaller than that of the top opening 30. The exterior sidewall 33 has a generally circular section, which does not substantially change in diameter along its length. The interior sidewall 33, having a generally circular section, continuously connects the top opening 30 and the bottom end 34 so that the surfaces defining the hollow interior 32 describe a receiver having an inverted, generally frusto-conical shape. In this embodiment the connector 12 is shown to be located on the top side 13 of the compression pad 10.

FIG. 7B is a section view on line 7B-7B in FIG. 7A whereby the connector 12 includes a top opening 30, an interior sidewall 31, a hollow interior 32, an exterior sidewall 33, and a closed bottom end, 34. This view more clearly illustrates the inverted, generally frusto-conical shape described by the interior surfaces defining the hollow interior 32, and further shows the connector 12 to be formed as an element of the substrate 11.

This detailed description of the invention is for illustrative purposes only. A reading by those skilled in the art will bring to mind various changes without departing from the spirit and scope of the invention.

We claim:

1) A compression pad, for use with vascular compression devices, which includes a substrate and a friction member; said substrate being formed of a first material which is rigid; said friction member being formed of a second material which has a friction coefficient greater than that of said first material; the friction member covering at least a portion of the surface of said compression pad.

2) The compression pad of claim 1 wherein the compression pad further includes an open notch extending from the outermost circumference of the compression pad generally towards the center of the compression pad, and a connector for connecting to and disconnecting from a vascular compression device.

3) The compression pad of claim 2 wherein said connector includes an exterior sidewall having a generally circular section and a hollow interior; said hollow interior including an opening having a generally circular circumference at its top end, and a closed bottom end having a generally circular circumference; said top end having a certain diameter and said bottom end having a diameter smaller than that of the top end; the interior sidewall of the hollow interior extending generally uniformly from the circumference of its bottom end to the circumference of its top end.

4) The compression pad of claim 1 wherein:
a) the compression pad further includes an upturned perimeter, a rim located on the topmost circumference of said upturned perimeter, and an open notch extending from said rim generally towards the center of the compression pad;
b) the friction member covering at least a portion of the bottom side of the compression pad.

5) The compression pad of claim 4 wherein:
a) the compression pad further includes a connector for connecting to and disconnecting from a vascular compression device;
b) said notch having a V shape with the widest portion of said V shape located at the rim of the compression pad and the narrow portion of the V shape pointed generally towards the center of the compression pad.

6) The compression pad of claim 5 wherein said connector includes an exterior sidewall having a generally circular section and a hollow interior; said hollow interior including an opening having a generally circular circumference at its top end, and a closed bottom end having a generally circular circumference; said top end having a certain diameter and said bottom end having a diameter smaller than that of the top end; the interior sidewall of the hollow interior extending generally uniformly from the circumference of its bottom end to the circumference of its top end.

7) The compression pad of claim 4 wherein:
a) the compression pad further having a generally round circumference;
b) said notch having a V shape with the widest portion of said V shape located at the rim of the compression pad and the narrow portion of the V shape pointed towards the center of the compression pad;
c) the substrate further including at least one selected from the group consisting of:
i) at least one foramen included in the substrate, in open conmmunication with the top and bottom surfaces of the substrate, wherein said bottom surface includes said upturned perimeter and said top surface is the surface opposite the bottom surface;
ii) at least one cavity with its opening in the bottom surface of the substrate;
iii) at least one recess formed into the bottom surface of the substrate;
iv) at least one groove traversing at least a portion of the circumference around the bottom surface including the upturned perimeter;
v) a connector for connecting to and disconnecting from a vascular compression device;
d) the friction member further covering at least a portion of the bottom side of the compression pad where said bottom side includes the bottom surface and the upturned perimeter; the friction member further attaching to the substrate by at least one of the means included in the group consisting of:
i) mechanical attachment by means of at least one top head, continuously composed of the same second material as forms the friction member, where the second material flows through at least one said foramen to form said top head on the top surface of the substrate during injection molding of the friction member onto the substrate; the top head further including a bottom surface resting on at least a portion of the top surface of the substrate;
ii) mechanical attachment by means of at least one post, continuously composed of the same second material as forms the friction member, where the second material flows through at least one said cavity to form said post during injection molding of the friction member onto the substrate; the post being contained in the cavity;
iii) mechanical attachment by means of at least one boss, continuously composed of the same second material as forms the friction member, where the second material flows into at least one said recess to form said boss and friction member within the recess during the injection molding of the friction member onto the substrate; at least a portion of the friction member being contained within the recess;
iv) mechanical attachment by means of at least one boss, continuously composed of the same second material as forms the friction member, where the second material flows into at least one said groove to form said boss on the upturned perimeter of the substrate during injection molding of the friction member onto the substrate;
v) at least some molecular cross-linking created between the first material and second material at the time of injection molding the friction member onto the substrate;
vi) at least some bonding created between the first material and second material at the time of injection molding the second material onto the first material;
vii) adhesive material placed on at least one of the adjoining surfaces of the friction member and the substrate prior to joining said surfaces together.

8) The compression pad of claim 7 wherein said connector includes an exterior sidewall having a generally circular section and a hollow interior; said hollow interior including an opening having a generally circular circumference at its top end, and a closed bottom end having a generally circular circumference; said top end having a certain diameter and said bottom end having a diameter smaller than that of the top end; the interior sidewall of the hollow interior extending generally uniformly from the circumference of its bottom end to the circumference of its top end.

9) A compression pad, for use with vascular compression devices, which includes a substrate and a friction member; said substrate being injection molded of a first material, which is rigid; said friction member being injection molded of a second material onto the substrate; said second material having a coefficient of friction higher than that of said first material.
